(19) United States
(12) Patent Application Publication
(10) Pub. No.: US 2025/0266162 A1
(43) Pub. Date: Aug. 21, 2025

(54) METHOD OF TARGETED MULTI-PANEL APPROACH AND TIERED A.I. USE FOR DIFFERENTIAL DIAGNOSIS AND PROGNOSIS

(71) Applicant: ARIZONA BOARD OF REGENTS ON BEHALF OF THE UNIVERSITY OF ARIZONA, Tucson, AZ (US)

(72) Inventors: Ruslan Rafikov, Tucson, AZ (US); Olga Rafikova, Tucson, AZ (US); Alexander Borovinskiy, Tucson, AZ (US)

(21) Appl. No.: 18/560,349

(22) PCT Filed: May 13, 2022
(86) PCT No.: PCT/US22/29270

§ 371 Date: Nov. 10, 2023

Related U.S. Application Data
(60) Provisional application No. 63/188,157, filed on May 13, 2021.

(51) Int. Cl. G16H 50/20; A61B 5/00; G16H 50/70

(52) U.S. Cl. CPC G16H 50/20; A61B 5/7267; G16H 50/70

(57) ABSTRACT

A diagnostic platform that enables multi-disease diagnostic panels which will help primary care physicians track the health status of patients as well as recognize disease early. The diagnostic platform implements a method of biomarker selection and tiered Artificial Intelligence (A.I.) approach comprising a multi-level machine/deep learning (ML/DL) system which is using multi-panels of biomarkers.

### CROSS-REFERENCES TO RELATED APPLICATIONS

This application claims benefit of U.S. Provisional Application No. 63/188,157 filed May 13, 2021, the specification of which is incorporated herein in its entirety by reference.

### STATEMENT REGARDING FEDERALLY SPONSORED RESEARCH OR DEVELOPMENT

This invention was made with government support under Grant Nos. HL133085 and HL132918, awarded by National Institutes of Health. The government has certain rights in the invention.

### FIELD OF THE INVENTION

The present invention features a diagnostic and prognostic platform of Artificial Intelligence (A.I.) assisted identification of chronic and acute conditions based on biomarker panels. Specifically, the diagnostic and prognostic platform will enable the use of multi-disease diagnostic panels which will help primary care physicians track the health status of patients as well as recognize the disease conditions.

### BACKGROUND OF THE INVENTION

Precision medicine tools can be applied to diagnose many chronic and acute disease conditions, including analysis of circulating proteins and metabolites. Cells and organs dynamically change their metabolic fluxes and profile of proteins secreted into circulation, reflecting both the transition from the normal health state to diseased and the severity level of disease progression. These changes in circulating biomarkers could be captured using mass spectrometry or other approaches and used to diagnose the disease or make prognostic decisions. The current challenges for using circulating disease biomarkers include low reproducibility, variability of detected biomarkers, and low statistical power due to non-targeted approaches to biomarkers identification.

Here, the present invention described the method of biomarkers selection and tiered A.I. use to overcome the current limitations for diagnostic or prognostic approaches. The tiered A.I. approach (single-tiered or multi-tiered) comprises a multi-level machine/deep learning ML/DL system that is using multi-panels of biomarkers. In the first tier, ML/DL algorithms or ensemble algorithms are trained to distinguish the changes in metabolomics/proteomic profiles induced by specific organs or cell types affected in particular pathological conditions. In the second tier, another trained A.I. model continues to sub-phenotype the disease. Extra tiers may be required to sub-phenotype different etiologies or co-morbidities. The tiered A.I. approach is utilizing specific multi-biomarkers panels from optimization by A.I. models with the expert-in-the-loop. Each organ or cell type requires a specific multi-biomarker panel to subphenotype the disease.

One aspect of the invention is that the biomarker panel used in each tier can be selected based on the results obtained in the previous tier. The first tier may indicate the particular organ or tissue type that is affected by a disease process. A panel of biomarkers relevant to that organ or tissue type would then be selected for the second tier. The second tier may indicate the disease that is present in that organ or tissue type. A disease-specific panel of biomarkers could then be selected for the third tier. The third tier may indicate the disease severity or progression and provide prognostic information. At each tier, the model performs the selection of the biomarker panel(s) for the next tier. There can be more than one panel selected at each tier because more than one disease may be indicated.

In some embodiments, the selection of biomarkers for the A.I.-tiered approach is a three-stage process. In the first stage, differences in biomarkers are detected between two or more tested disease conditions, including healthy individuals. In the second stage, differently expressed circulating biomarkers are refined by removing exogenous substances and manual selection of biomarkers that involve the disease pathology of the distinguished organs/cell types. In the third stage, ML/DL models are utilized to refine further the biomarkers panel based on feature importance calculated. This approach includes iteration-based optimization of the ML/DL model performance using constantly refined biomarkers panels. This targeted biomarkers multi-panel selection coupled with an A.I.-tiered methodology will be utilized to differentially diagnose disease conditions, track health/disease status, make the disease prognosis, perform routine screening, identify patients at risk, and monitor and evaluate the effectiveness of therapy.

### BRIEF SUMMARY OF THE INVENTION

It is an objective of the present invention to provide computer platforms and methods of use that allow for the diagnosis and prognosis of patients with a variety of diseases, as specified in the independent claims. Embodiments of the invention are given in the dependent claims. Embodiments of the present invention can be freely combined with each other if they are not mutually exclusive.

The present invention features a computer-implemented method for diagnosing a subject with a disease. The method may also include prognosing the subject with the disease, medical screening, monitoring therapy efficacy, or a combination thereof. In some embodiments, the method comprises inputting into a computer system quantitative data (or expression data) of a panel of biomarkers in a biological sample obtained from the subject. In some embodiments, the computer system may comprise a processor capable of executing computer-readable instructions, and a memory component capable of storing a plurality of computer-readable instructions able to be executed by the processor. In some embodiments, the method comprises determining the biomarkers multi-panel that can distinguish healthy patients from diseases that affect different organs or cell types using three stage biomarkers selection based on 1) statistical significance, 2) pathology of disease and 3) feature selection optimization by machine learning or deep learning algorithms executed on a plurality of clinical parameters. In some embodiments, the machine learning classifier has been trained using quantitative data of a panel of biomarkers from subjects having the disease and from control subjects that do not have disease. In some embodiments, the method comprises determining a second biomarkers panel that can implement machine learning and deep learning algorithms to sub-phenotype the disease of the organ affected identified aforementioned step. In some embodiments, the method comprises determining a third biomarkers panel that can implement machine learning and deep learning algorithms to identify specific etiology or comorbidities of the disease of the organ affected identified in the aforementioned step. In some embodiments, the method comprises diagnosing the subject if the quantitative data of the panel of biomarkers in the biological sample obtained from the subject is correlated by the computer system using tiered panels and machine learning and deep learning algorithms to produce risk scores for the one or more diseases.

The present invention may also feature a non-transitory, computer-readable medium having computer-executable instructions for causing a processor to execute a method for diagnosing a subject with a disease. In some embodiments, the method comprises determining whether the quantity of a panel of metabolic biomarkers in a biological sample obtained from the subject is indicative of the disease using a trained machine learning classifier for distinguishing subjects with different diseases and without the disease. In some embodiments, the machine learning classifier has been trained using quantitative data of a panel of metabolic biomarkers from subjects having the disease and from control subjects that do not have the disease. In some embodiments, the method comprises diagnosing the subject if the quantitative data is correlated to be indicative of the disease.

The present invention may feature a kit for diagnosing a subject with a disease. In some embodiments, the kit comprises one or more reference metabolic biomarker panels; and a non-transitory, computer-readable medium as described herein. In some embodiments, quantitative data of a panel of metabolic biomarkers in a biological sample obtained from the subject is inputted into a computer that executes the computer-executable instructions of the non-transitory, computer-readable medium. In some embodiments, the subject is diagnosed with the disease when the quantitative data of the panel of metabolic biomarkers in the biological sample obtained from the subject is correlated with the one or more reference metabolic biomarker panels by the computer to be indicative of disease.

The present invention may also feature a non-transitory, computer-readable medium having computer-executable instructions for training a multi-label machine learning model to identify disease biomarkers in a patient. In some embodiments, the computer-executable instructions comprise computationally selecting one or more profiles, wherein each profile is selected from a group comprising metabolomic profiles, proteomic profiles, or a combination thereof. In other embodiments, the computer-executable instructions comprise computationally selecting, for each profile of the one or more profiles, one or more change-disease relationships between a change to the profile and one or more diseases that induce the change. In some embodiments, the computer-executable instructions comprise providing a structural model for each change-disease; and processing, by at least a first tier of the machine learning model, each structural model such that the machine learning model is trained to identify, based on a change to a profile of the patient, the one or more diseases that induced the change.

The present invention may additionally feature a computer-implemented method for diagnosing a subject with a disease. In some embodiments, the method comprises inputting into a computer system quantitative data of a panel of biomarkers in a biological sample obtained from the subject. In some embodiments, the method comprises determining the biomarkers multi-panel that can distinguish healthy patients from diseases that affect different organs or cell types using three-stage biomarkers selection based on 1) statistical significance, 2) pathology of disease and 3) feature selection optimization by machine learning or deep learning algorithms executed on a plurality of clinical parameters. In some embodiments, the machine learning classifier has been trained using quantitative data of a panel of biomarkers from subjects having the disease and from control subjects that do not have the disease. In some embodiments, the method comprises diagnosing the subject if the quantitative data of the panel of biomarkers in the biological sample obtained from the subject is correlated by the computer system using tiered panels and machine learning and deep learning algorithms to be indicative of the disease. In some embodiments, the method comprises predicting, by the plurality of biomarkers panels and the diagnosis, a disease mortality of the subject up to a number of years with at least 35% accuracy.

One of the unique and inventive technical features of the present invention is the use of multi-panel biomarkers. Without wishing to limit the invention to any theory or mechanism, it is believed that the technical feature of the present invention advantageously provides for the ability to predict the mortality of the one or more diseases with higher than 60% accuracy, which cannot be done with other risk-score assessments. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWINGS

The features and advantages of the present invention will become apparent from a consideration of the following detailed description presented in connection with the accompanying drawings in which:

FIG. 1A shows a non-limiting example of how multiple panels can be used to diagnose various diseases. In some embodiments, multiple panels may be used to distinguish between similar diseases.

FIG. 1B shows a non-limiting example of a computer workflow as described herein.

FIGS. 2A and 2B show a redox-based clustering of control and PAH plasma samples in each gender. Principal component analysis (PCA) of cytokines that were differentially expressed in two extreme redox conditions, the most and the least oxidized, revealed the clustering of PAH samples with Low-oxidative-reductive potential (ORP), High-ORP, and control samples in each gender. FIG. 2A shows that, in males, IL-1b, a pro-inflammatory cytokine, showed the highest involvement in separating patients with High-ORP from controls. MIP-1a, G-CSF, IL-6, IL-1ra, VEGF, IL-10, and Eotaxin exhibited influence on clustering of patients with Low-ORP.

FIG. 2B shows that, in females, not only IL-1b, but also IL-2, IL-13, IL-7, and IL-17 contributed to the clustering of High-ORP samples. The Low-ORP group's separation was driven by Eotaxin, IL-8, IL-10, MIP-1a, IFNg, VEGF, IL-1ra, and MCP-1. Overall, High-ORP clustering is mediated by pro-inflammatory cytokines, and Low-ORP—by proliferative and anti-inflammatory pathways.

FIGS. 3A and 3B show the sex-specific separation of PAH patient cohort based on cytokine profiles. FIG. 3A shows a stochastic gradient descent machine learning algorithm trained on sex-specific cytokine profiles was able to distinguish males and females with 87-90% accuracy, confirming the presence of distinct sex-based profiles in cytokine expression identifiable by machine learning models. FIG. 3B shows cytokines IL-1ra, IL-2, IL-12, IFNg, IP10, and IL-8 were identified as the most potent contributors in the differentiation of male vs. female cytokine profiles. Information gain values indicate the ranking.

FIGS. 4A and 4B show a redox-specific separation of the PAH patient cohort based on cytokine profiles. FIG. 4A shows a support vector machine trained on redox-specific profiles in each sex group distinguished between High-ORP and Low-ORP plasma samples with 95-100% accuracy. FIG. 4B shows that the data confirm that the difference in the redox environment triggers the distinct patterns of cytokine expression that could be accurately recognized by machine learning models. MCP-1, VEGF, IL-1ra, Eotaxin, IL-1b, and IL-10 were identified as the primary contributors to the redox-based profiling in females, whereas VEGF, IL-10, IL-6, IFNg, IL-1ra were responsible for the redox-based separation in males. Information gain values indicate the ranking.

FIGS. 5A, 5B, 5C, 5D, and 5E show that a cytokine profile, but not clinical parameters, predicts PAH patient mortality. FIG. 5A shows the Kaplan-Meier estimates of five-year survival for each gender were compared by log-rank test. FIG. 5B shows the Naïve Bayes machine learning algorithm trained on the cytokine profiles predicted mortality in the total PAH patient cohort with 85% accuracy. The cytokines with the highest rank for prediction of patient mortality were identified as IL-6, IL-7, IL-1b, and IL-4. FIG. 5C shows the ORP was identified as one of the highly ranked factors responsible for predicting patient mortality. FIG. 5D shows that the same machine-learning algorithm applied for the primary clinical parameters predicted patient mortality with 35% accuracy, although it showed a comparable accuracy for predicting patient survival. FIG. 5E shows that the PVR, 6MWD, and mPAP showed the highest among the clinical parameters rank for prediction of the outcomes in PAH patients. Information gain values indicate the ranking.

FIG. 6 shows a Redox-based profile of circulating cytokines. The contribution of the redox status was evaluated by comparing the levels of circulating cytokines in Controls (first boxplot in each graph) vs. 25% of least oxidized samples (lowest ORP quartile, second boxplot) vs. 25% of most oxidized samples (highest ORP quartile, third boxplot) in each sex group. Boxplots are presented only for redox-sensitive cytokines (25% or 75% quartile is significantly different vs. Controls). P-value is indicated for the Student t-test.

### DETAILED DESCRIPTION OF THE INVENTION

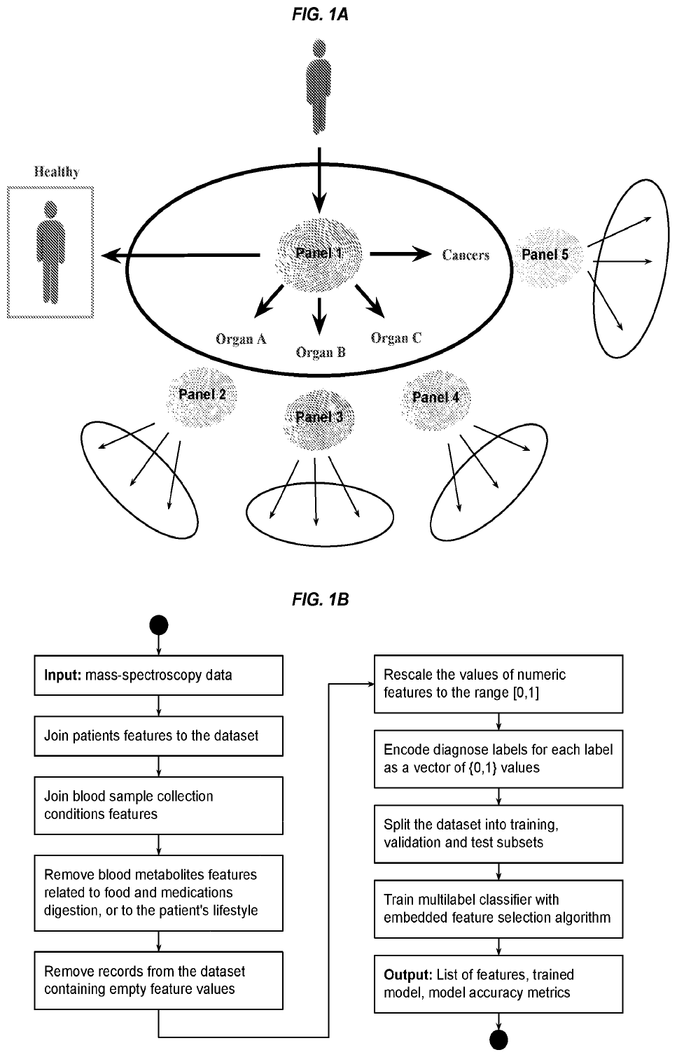

Referring now to FIGS. 1A-6, the present invention features computer platforms and methods of use that allow for the early diagnosis of patients with a variety of diseases.

In some embodiments, the present invention features a computer-implemented method for diagnosing a subject with a disease. In some embodiments, the method comprises inputting into a computer system quantitative data of a panel of biomarkers in a biological sample obtained from the subject. In some embodiments, the computer system may comprise a processor capable of executing computer-readable instructions, and a memory component capable of storing a plurality of computer-readable instructions able to be executed by the processor. In some embodiments, the method comprises determining the biomarkers multi-panel that can distinguish healthy patients from diseases that affect different organs or cell types using three-stage biomarkers selection based on 1) statistical significance, 2) pathology of disease and 3) feature selection optimization by machine learning or deep learning algorithms executed on a plurality of clinical parameters. In some embodiments, the machine learning classifier has been trained using quantitative data of a panel of biomarkers from subjects having the disease and from control subjects that do not have the disease. In some embodiments, the method comprises determining a second biomarkers panel that can implement machine learning and deep learning algorithms to sub-phenotype the disease of the organ affected identified aforementioned step. In some embodiments, the method comprises determining a third biomarkers panel that can implement machine learning and deep learning algorithms to identify specific etiology or comorbidities of the disease of the organ affected identified in the aforementioned step. In some embodiments, the method comprises diagnosing the subject if the quantitative data of the panel of biomarkers in the biological sample obtained from the subject is correlated by the computer system using tiered panels and machine learning and deep learning algorithms to be indicative of the disease.

In some embodiments, the present invention features a computer-implemented method for diagnosing and prognosing a subject with a disease. In some embodiments, the method comprises inputting into a computer system quantitative data of a panel of biomarkers in a biological sample obtained from the subject. In some embodiments, the computer system may comprise a processor capable of executing computer-readable instructions, and a memory component capable of storing a plurality of computer-readable instructions able to be executed by the processor. In some embodiments, the method comprises analyzing the quantitative data with machine learning or deep learning models or their ensembles. In other embodiments, the method comprises using a first-tier biomarker multi-panel to distinguish healthy subjects from subjects with a disease that affects different organs or cell types. In some embodiments, the subject with a disease may have multiple diseases. In some embodiments, the biomarker multi-panel was previously determined by using a three-stage biomarkers selection based on 1) statistical significance, 2) pathology of disease and 3) feature selection optimization by machine learning or deep learning algorithms executed on a plurality of clinical parameters. In some embodiments, the machine learning, deep learning, or ensemble classifier has been trained using quantitative data of a panel of biomarkers from subjects having the disease and from control subjects that do not have the disease. In some embodiments, the method comprises determining and using a second-tier biomarkers panel that can implement machine learning and deep learning algorithms to sub-phenotype the disease of the organ or the cell type affected identified above. In some embodiments, the method comprises determining and using a third-tier biomarkers panel that can implement machine learning and deep learning algorithms to identify specific etiology or comorbidities of the disease of the organ or the cell type affected identified above. In some embodiments, the method comprises diagnosing or prognosing the subject if the quantitative data of the panel of biomarkers in the biological sample obtained from the subject is correlated by the computer system using tiered panels and machine learning and deep learning algorithms to be indicative of the disease.

In other embodiments, the method may further comprise steps for preparing the quantitative data of the panel of metabolic biomarkers for inputting into the computer system. In some embodiments, the steps comprise 1) labeling the quantitative data with one or more confirmed diagnoses of a pathological condition, 2) applying a plurality of characteristics of the patient to the quantitative data, 3) balancing the dataset through the exclusion of data that does not correspond to a disease biomarker, the addition of multiple-use data points, or a combination thereof; and 4) scaling the dataset to a fixed range.

In some embodiments, the trained machine learning and deep learning algorithms comprise linear regression, logistic regression, decision tree, support vector machine, Naive Bayes, K nearest neighbors, K-Means, random forest, artificial neural networks, or a combination thereof.

In some embodiments, a biological sample may comprise plasma, serum, cerebrospinal fluid, lymph, bronchial lavage fluid, or urine from the subject. The sample may be spiked with internal standards so as to calibrate analysis. As a non-limiting example, a biological sample may be combined with a known amount of a known analyte such as isotope (D, 13C, 15N, 17O and other)-labeled metabolites, molecules and compositions.

In some embodiments, the quantitative data of the panel of metabolic biomarkers is determined using standard clinical chemistry techniques, protein analytic techniques, nucleic acid techniques, and/or analytical techniques suitable for metabolite analysis (e.g., Mass spectrometry (MS), gas chromatography (GC) coupled to mass spectrometry (GC-MS), liquid chromatography-mass spectrometry (LCMS) or other mass spectrometry methods, or nuclear magnetic resonance (NMR)).

In some embodiments, the input datasets contain MS data from biological samples (e.g. a blood plasma sample) from a patient. In some embodiments, the sample is labeled with a confirmed diagnosis. In other embodiments, the sample is not labeled with a diagnosis. In certain embodiments, multiple diagnoses may be assigned to the sample (multi-label classification). In other embodiments, samples may have incomplete sets of labels (missing label problem).

In some embodiments, the dataset may also include gender, age, race and ethnicity information from the patient, time and date of sample collection, patient's condition at the time of the sample collection (fasting/non-fasting), data on the mass-spec device used for sample processing, etc. In some embodiments, the clinical parameters comprise sex, plasma redox status, and cytokine levels.

In some embodiments, the plurality of characteristics comprises gender, age, race, ethnicity, time and date of sample collection, and patient condition at the time and date of sample collection. In other embodiments, the excluded data comprises metabolites associated with the consumption of certain food or drugs, redundant metabolites, and metabolites that contribute to noise.

In some embodiments, the multiple-use data points comprise randomly picked data points with an underrepresented label for the purpose of filling in missing metabolite data points. In some embodiments, the dataset is scaled to a range of [0, 1].

In other embodiments, the present invention utilizes metabolites comprising carbohydrates, amino acids, fatty acids, and/or nucleotides and their derivatives. In some embodiments, the metabolites comprise carbohydrates, amino acids, fatty acids, and/or nucleotides and their intermediates or derivatives.

In some embodiments, the present invention may feature a non-transitory, computer-readable medium having computer-executable instructions for causing a processor to execute a method for diagnosing a subject with a disease. In some embodiments, the method comprises determining whether the quantitative data of a panel of metabolic biomarkers in a biological sample obtained from the subject is indicative of the disease using a trained machine learning classifier for distinguishing subjects with different diseases and without the disease. In some embodiments, the machine learning classifier has been trained using quantitative data of a panel of metabolic biomarkers from subjects having the disease and from control subjects that do not have the disease. In some embodiments, the method comprises diagnosing the subject if the quantitative data is correlated to be indicative of the disease.

In other embodiments, the present invention may feature a kit for diagnosing a subject with a disease. In some embodiments, the kit comprises one or more reference metabolic biomarker panels; and a non-transitory, computer-readable medium as described herein. In some embodiments, quantitative data of a panel of metabolic biomarkers in a biological sample obtained from the subject is inputted into a computer that executes the computer-executable instructions of the non-transitory, computer-readable medium. In some embodiments, the subject is diagnosed with the disease when the quantitative data of the panel of metabolic biomarkers in the biological sample obtained from the subject is correlated with the one or more reference metabolic biomarker panels by the computer to be indicative of disease.

The present invention may feature a non-transitory, computer-readable medium having computer-executable instructions for training a multi-label machine learning model to identify disease biomarkers in a patient. In some embodiments, the computer-executable instructions comprise computationally selecting one or more profiles, wherein each profile is selected from a group comprising metabolomic profiles, proteomic profiles, or a combination thereof. In other embodiments, the computer-executable instructions comprise computationally selecting, for each profile of the one or more profiles, one or more change-disease relationships between a change to the profile and one or more disease biomarkers that induce the change. In some embodiments, embodiments, the computer-executable instructions comprise providing a structural model for each change-disease; and processing, by at least a first tier of the machine learning model, each structural model such that the machine learning model is trained to identify, based on a change to a profile of the patient, the one or more disease biomarkers that induced the change.

In some embodiments, the non-transitory, computer-readable medium may further comprise computer-executable instructions. In some embodiments, the computer-executable instructions comprise computationally selecting, for each disease biomarker selected, one or more disease-etiology relationships between the disease biomarker and one or more etiologies of the disease biomarker. In other embodiments, the computer-executable instructions comprise providing a structural model for each disease-etiology relationship. In some embodiments, the computer-executable instructions comprise processing, by at least a second tier of the machine learning model, each structural model such that the machine learning model is trained to identify, based on the one or more changes to the profile of the patient and the one or more disease biomarkers identified in the patient, the one or more etiologies of the one or more disease biomarkers.

In other embodiments, the aforementioned non-transitory, computer-readable medium further comprises computer-executable instructions comprising computationally selecting, for each disease biomarker selected, one or more disease-comorbidity relationships between the disease biomarker and one or more comorbidities associated with the disease biomarker. In other embodiments, the computer-executable instructions comprise providing a structural model for each disease-comorbidity relationship. In some embodiments, the computer-executable instructions comprise processing, by at least a second tier of the machine learning model, each structural model such that the machine learning model is trained to identify, based on the one or more changes to the profile of the patient and the one or more disease biomarkers identified in the patient, the one or more comorbidities of associated with the one or more disease biomarkers.

In some embodiments, the aforementioned non-transitory, computer-readable medium further comprises computer-executable instructions comprising computationally selecting one or more exogenous substances that cause a change to the profile of the patient that simulates a disease biomarker. In other embodiments, the computer-executable instructions comprise computationally selecting one or more biomarker-organ relationships between a disease biomarker and an affected organ associated with the disease biomarker. In some embodiments, the computer-executable instructions may comprise providing a structural model for each biomarker-organ relationship. In some embodiments, the comprising computer-executable instructions further comprise processing, by at least a second tier of the machine learning model, each exogenous substance and each structural model such that the machine learning model is trained to refine the one or more disease biomarkers produced by at least the first tier by removing disease biomarkers caused by the one or more exogenous substances and selecting one or more disease biomarkers based on affected organs of the patient.

In other embodiments, the aforementioned non-transitory, computer-readable medium further comprises computer-executable instructions. In some embodiments, the computer-executable instructions comprise generating a set comprising the one or more disease biomarkers selected ordered by feature importance and processing, by at least a third tier of the machine learning model, the set of disease biomarkers ordered by feature importance such that the machine learning model is trained to further refine the one or more disease biomarkers produced by at least the second tier by removing disease biomarkers with low feature importance.

The present invention may additionally feature a computer-implemented method for diagnosing a subject with a disease. In some embodiments, the method comprises inputting into a computer system quantitative data of a panel of biomarkers in a biological sample obtained from the subject. In some embodiments, the method comprises determining the biomarkers multi-panel that can distinguish healthy patients from diseases that affect different organs or cell types using three-stage biomarkers selection based on 1) statistical significance, 2) pathology of disease and 3) feature selection optimization by machine learning or deep learning algorithms executed on a plurality of clinical parameters. In some embodiments, the machine learning classifier has been trained using quantitative data of a panel of biomarkers from subjects having the disease and from control subjects that do not have the disease. In some embodiments, the method comprises diagnosing the subject if the quantitative data of the panel of biomarkers in the biological sample obtained from the subject is correlated by the computer system using tiered panels and machine learning and deep learning algorithms to be indicative of the disease. In some embodiments, the method comprises predicting, by the plurality of biomarkers panels and the diagnosis, a PAH mortality of the subject up to a number of years with at least 35% accuracy.

In some embodiments, the method further comprises determining a second biomarkers panel that can implement machine learning and deep learning algorithms to sub-phenotype the disease of the organ affected identified. In other embodiments, the method further comprises determining a third biomarkers panel that can implement machine learning and deep learning algorithms to identify specific etiology or comorbidities of the disease of the organ affected identified.

In some embodiments, the quantitative data of the panel of biomarkers is determined using standard clinical chemistry techniques, protein analytic techniques, nucleic acid techniques, and/or analytical techniques suitable for metabolite analysis. In some embodiments, the techniques comprise gas chromatography (GC) coupled to mass spectrometry (GC-MS), liquid chromatography-mass spectrometry (LC-MS), other mass spectrometry methods or nuclear magnetic resonance (NMR).

In some embodiments, predicting mortality comprises executing a Naïve Bayes algorithm on the plurality of clinical parameters.

In some embodiments, the number of years is up to 5 years. In some embodiments, the number of years is up to 6 years. In some embodiments, the number of years is up to 7 years. In some embodiments, the number of years is up to 8 years. In some embodiments, the number of years is up to 9 years. In some embodiments, the number of years is up to 10 years. In some embodiments, the number of years is up to 4 years. In some embodiments, the number of years is up to 3 years. In some embodiments, the number of years is up to 2 years.

In some embodiments, the list of metabolites found in the patient's samples is screened against the Human Metabolome Database. In other embodiments, specific metabolites associated with the consumption of certain food, or drugs are excluded from the dataset. In other embodiments, redundant metabolites are excluded. In some embodiments, metabolites that contribute to noise are excluded.

In some embodiments, the datasets are balanced to have the same number of samples with different labels (diagnoses) by randomly picking samples with an underrepresented label and adding their copies to the dataset (Standard procedure).

In some embodiments, any missing data points are replaced with the mean value calculated from the current metabolite values from other samples (Standard procedure). In other embodiments, records with missing data points are excluded from consideration.

In some embodiments, the values in the dataset are scaled to the range [0,1] (Standard procedure). In other embodiments, the labels are encoded into vectors containing 0/1 values. Each label is mapped to a specific position in the vector. In some embodiments, the value 1 is assigned at this position if the sample is labeled with this diagnosis, 0 otherwise. (Standard procedure).

In preferred embodiments, 20% of the samples are randomly assigned to the test dataset. In other embodiments, 10% of the samples are randomly assigned to the test dataset. In some embodiments, 30% of the samples are randomly assigned to the test dataset. In other embodiments, the remaining records are split into multiple subsets and a cross-validation technique is used to train multiple models and average the prediction results across the models.

In some embodiments, the 80% of the samples are split into multiple subsets and a cross-validation technique is used to train multiple models and average the prediction results across the models. In some embodiments, the 90% of the samples are split into multiple subsets and a cross-validation technique is used to train multiple models and average the prediction results across the models. In some embodiments, the 70% of the samples are split into multiple subsets and a cross-validation technique is used to train multiple models and average the prediction results across the models.

In some embodiments, the quality of the trained machine model may be measured via a multi-label accuracy. In some embodiments, multi-label accuracy measures the average ratio of correctly classified labels to the total number of labels in the predicted and the true label sets. The accuracy score is the average score across all test instances. It takes a value in the range of zero to one (inclusive), with an optimal value of one.

In other embodiments, samples may be measured via a 0/1 subset accuracy. In some embodiments, a 0/1 subset accuracy measures the fraction of instances whose labels are perfectly predicted. It takes a value in the range of zero to one (inclusive), with an optimal value of one.

In further embodiments, the quality of the trained machine learning model may be measured via Hamming loss. In some embodiments, a Hamming loss measures the average fraction of misclassified labels across all test instances. It takes a value in the range of zero to one (inclusive), with an optimal value of zero.

In some embodiments, the trained machine learning classifiers are the machine learning/deep learning algorithms including logistic regression, neural network, and other algorithms. As used herein, “a machine learning classifier” utilizes some training data to train a model to predict the class (a disease) or multiple classes (a set of diseases) with given input variables (quantitative data of metabolic biomarkers).

In some embodiments, the present invention may include a processor in communication with various elements of hardware. In some embodiments, the processor includes one or more processors configured to implement a set of instructions corresponding to any of the methods disclosed herein. In other embodiments, the processor can be configured to implement a set of instructions (stored in the memory of hardware or sub-system) to provide a correlation between the quantitative data and a particular disease. In other embodiments, a sub-system can include hardware and software capable of facilitating the processing of data generated by hardware, in conjunction with, or as a substitute for, the processing that is normally handled by the processor.

In some embodiments, the diagnostic accuracy of the computer system is 100%. In some embodiments, the diagnostic accuracy of the computer system is at least 99%. In some embodiments, the diagnostic accuracy of the computer system is at least 98%. In some embodiments, the diagnostic accuracy of the computer system is at least 95%. In some embodiments, the diagnostic accuracy of the computer system is at least 90%. In some embodiments, the diagnostic accuracy of the computer system is 85%. In some embodiments, the diagnostic accuracy of the computer system is at least 80%. Without wishing to limit the present invention to any particular theory or mechanism, it is believed that diagnostic accuracy is a function of both the sensitivity and the selectivity of the system. As non-limiting examples, the sensitivity of the system may be at least 50, 55, 60, 65, 70, 75, 80, 85, 90, 95, or 99 percent and the selectivity of the system may be at least 90, 91, 92, 93, 94, 95, 96, 97, 98, or 99 percent.

In some embodiments, the present invention includes a computer system that can execute the methods for diagnosing a disease as described herein. In some embodiments, the invention employs a computer device or computer-implemented method having one or more processors and at least one memory, the at least one memory storing non-transitory computer-readable instructions for execution by the one or more processors to cause the one or more processors to execute instructions (or stored data) in one or more modules. Alternatively, the instructions may be stored in a non-transitory computer-readable medium or computer-usable medium. In some embodiments, a computer system can include a desktop computer, a laptop computer, a tablet, or the like and can include digital electronic circuitry, firmware, hardware, memory, a computer storage medium, a computer program, a processor (including a programmed processor), or the like. The computing system may include a desktop computer with a screen and a tower. The computing system may also include a cloud computing platform, such as Amazon AWS, Microsoft Azure, Google Cloud Platform, or the like.

Any methods, devices, and materials similar or equivalent to those described herein can be used in the practice of this invention. In some aspects, the methods of the present invention described herein are performed in vitro. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only and in no way limit the invention described herein.

The term “processor” encompasses all kinds of apparatus, devices, and machines for processing data, including by way of example a programmable microprocessor, a computer, a system on a chip, multiple ones, or combinations, of the foregoing. The apparatus can include special purpose logic circuitry, e.g., an FPGA (field-programmable gate array) or an ASIC (application-specific integrated circuit). The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing, and grid computing infrastructures. The processor may include one or more processors of any type, such as central processing units (CPUs), graphics processing units (GPUs), special-purpose signal or image processors, and field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other units suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures, disclosed in this specification and their structural equivalents, or in combinations of one or more of them. Embodiments described in this specification can be implemented as one or more computer programs, i.e., one or more modules of computer program instructions encoded on a computer storage medium for execution by, or to control the operation of, data processing apparatus. Any of the modules described herein may include logic that is executed by the processor(s). “Logic,” as used herein, refers to any information having the form of instruction signals and/or data that may be applied to affect the operation of a processor. Software is an example of logic. Logic may be formed from signals stored on a computer-readable medium such as memory that, in an exemplary embodiment, may be a random access memory (RAM), read-only memories (ROM), erasable/electrically erasable programmable read-only memories (EPROMS/EEPROMS), flash memories, etc. Logic may also comprise digital and/or analog hardware circuits, for example, hardware circuits comprising logical AND, OR, XOR, NAND, NOR, and other logical operations. Logic may be formed from combinations of software and hardware. On a network, logic may be programmed on a server or a complex of servers. A particular logic unit is not limited to a single logical location on the network. Moreover, the modules need not be executed in any specific order. Each module may call another module when needed to be executed.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or can be included in, one or more separate physical components or media (e.g., multiple CDs, disks, or other storage devices). The operations described in this specification can be implemented as operations performed by a data processing apparatus on data stored on one or more computer-readable storage devices or received from other sources.

Program code embodied on a computer-readable medium may be transmitted using any appropriate medium, including but not limited to wireless, wireline, optical fiber cable, R.F, etc., or any suitable combination of the foregoing. Computer program code for carrying out operations for aspects of the present disclosure may be written in any combination of one or more programming languages, including object-oriented programming languages such as Python, Java, Smalltalk, C++, or the like, and conventional procedural programming languages, such as the “C” programming language or similar programming languages. The program code may execute entirely on the user's computer, partly on the user's computer, as a stand-alone software package, partly on the user's computer, partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed, and apparatus can also be implemented as special purpose logic circuitry, e.g., an FPGA (field-programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory, or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media, and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

One or more computing devices such as desktop computers, laptop computers, tablets, smartphones, servers, application-specific computing devices, or any other type(s) of the electronic device(s) may be capable of performing the techniques and operations described herein. In some embodiments, the system may be implemented as a single device. In other embodiments, the system may be implemented as a combination of two or more devices together. For example, the system may include one or more server computers and one or more client computers communicatively coupled to each other via one or more local-area networks and/or wide-area networks such as the Internet.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light-emitting diode) display, or OLED (organic light-emitting diode) display, for displaying information to the user. Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user: for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what is referred to as “command line interfaces” (often referred to as CLI's). CLIs typically provide a text-based interaction between an application and a user. Typically, command-line interfaces present output and receive input as lines of text through display devices. For example, some implementations may include what is referred to as a “shell” such as Unix Shells known to those of ordinary skill in the related art, or Microsoft Windows Powershell that employs object-oriented type programming architectures such as the Microsoft .NET framework.

Those of ordinary skill in the related art will appreciate that interfaces may include one or more GUIs, CLIs, or a combination thereof. A processor may include a commercially available processor such as a Celeron, Core, or Pentium processor made by Intel Corporation, a SPARC processor made by Sun Microsystems, an Athlon, Sempron, Phenom, Ryzen or Opteron processor made by AMD Corporation, or it may be one of other processors that are or will become available. Some embodiments of a processor may include a multi-core processor and/or be enabled to employ parallel processing technology in a single or multi-core configuration. For example, a multi-core architecture typically comprises two or more processor “execution cores”. Each execution core may perform as an independent processor that enables the parallel execution of multiple threads. In addition, those of ordinary skill in the related field will appreciate that a processor may be configured in what is generally referred to as 32 or 64-bit architectures, or other architectural configurations now known or that may be developed in the future.

A processor typically executes an operating system, which may be, for example, a Windows type operating system from the Microsoft Corporation: the Mac OS X operating system from Apple Computer Corp.; a Unix or Linux-type operating system available from many vendors, or what is referred to as an open-source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, communication control, and related services, all in accordance with known techniques.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks). For example, the network can include one or more local area networks. The computing system can include any number of clients and servers. A client and server are generally remote from each other and typically interact through a communication network. The relationship between client and server arises by virtue of computer programs running on the respective computers and having a client-server relationship with each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Also, a computer may include one or more library files, experiment data files, and an internet client stored in system memory. For example, experiment data could include data related to one or more experiments or assays, such as detected signal values, or other values associated with the biomarker quantitative data. Additionally, an internet client may include an application enabled to access a remote service on another computer using a network and may for instance comprise what is generally referred to as “Web Browsers”. In the present example, some commonly employed web browsers include Microsoft Internet Explorer available from Microsoft Corporation, Mozilla Firefox from the Mozilla Corporation, Safari from Apple Computer Corp., Google Chrome from the Google Corporation, or other types of web browsers currently known in the art or to be developed in the future. Also, in the same or other embodiments, an internet client may include or could be an element of specialized software applications enabled to access remote information via a network such as a data processing application for biological applications.

A network may include one or more of the various types of networks known to those of ordinary skill in the art. For example, a network may include a local or wide area network that may employ what is commonly referred to as a TCP/IP protocol suite to communicate. A network may include a network comprising a worldwide system of interconnected computer networks that is commonly referred to as the internet or could also include various intranet architectures. Those of ordinary skill in the related arts will also appreciate that some users in networked environments may prefer to employ what are generally referred to as “firewalls” (also sometimes referred to as Packet Filters, or Border Protection De-vices) to control information traffic to and from hardware and/or software systems. For example, firewalls may comprise hardware or software elements or some combination thereof and are typically designed to enforce security policies put in place by users, such as for instance network administrators, etc.

When executed, instructions (which may be stored in the memory) cause at least one of the processors of the computer system to receive an input, which is quantitative data of a panel of metabolic biomarkers in a biological sample obtained from the subject i. Once the necessary inputs are provided, a module is then executed to derive object features and context features and to calculate object feature metrics and context feature metrics. The object feature metrics and context feature metrics are provided to a trained end classifier, which classifies the object and provides an output to the user. The output may be to a display, a memory, or any other means suitable for the art.

### EXAMPLE

The following is a non-limiting example of the present invention. It is to be understood that said example is not intended to limit the present invention in any way. Equivalents or substitutes are within the scope of the present invention.

### Methods

Patient cohorts: PAH and control subjects were prospectively recruited by the University of Arizona (UA). All subjects provided written consent to participate in this study with the approval of the UA institutional human subjects review board. Peripheral venous blood was collected during outpatient clinic visits or right heart catheterization and stored at the University of Arizona Biobank. Care was taken to standardize blood sample collection, preparation, and storage at −80° C.

141 PAH patients (41 males and 100 females) who met the World Symposium of PH Group 1 criteria (30) and 50 healthy subjects (29 males and 21 females) were used in this study for redox and cytokine profiling. Clinical data were extracted from the electronic medical record: 6-minute walk distance (6MWD), brain natriuretic peptide (BNP), and functional class (FC) tests were selected based on the completion of assessment date closest to the date of right heart catheterization. The outcome of time to death was assessed during the five-year period that followed blood sampling. The cohort characteristics at blood sampling are presented in Table 1.

Redox parameters evaluation: Oxidation-reduction potential (ORP) was measured in 30 μL of patient samples electrochemically using RedoxSys® Diagnostic System (Aytu BioScience Inc., Englewood, CO), the diagnostic platform that measures ORP in body fluids as described in the manufacturer's protocol.

Cytokine multiplex assay: The Bio-Plex multiplex immunoassay platform permits high throughput identification of proteins in the biological samples using premade or custom-made panels. The Bio-Plex Pro Human Cytokine Group1 Panel 27-Plex (Bio-Rad, #M5000KCAF0Y) was used for the analysis of cytokines, chemokines, and growth factors in human plasma of healthy and PAH subjects. Bead-based assay permits the detection of 27 different types of cytokine, chemokine, or growth factor target in a single well of a 96-well microplate. The assay was performed according to the manufacturer's protocol. Briefly, human plasma was diluted two-fold with Bio-Plex sample diluent and added to beads covalently coupled to antibodies against 27 targets. After 30 minutes of incubation on a shaker at room temperature, beads were washed, and biotinylated detection antibodies were added for 30 minutes under the same conditions. After a 3-time wash, streptavidin-phycoerythrin (streptavidin-PE) complex was added to bind to the biotinylated detection antibodies for 10 minutes at room temperature. The plate was processed on the Bio-Plex instrument immediately. Data Acquisition at low PMT, RPI setting and Analysis Data was performed using the Bio-Plex 200 System (Bio-Rad).

Principal component analysis: Principal component analysis (PCA) was applied to the controls and PAH patients to visualize high-dimensional data clustering. To analyze and plot the data set, the Orange software package (version 3.26) was utilized. Cohorts were disaggregated by sex, and PCA was done on cytokines that showed redox-specific expression profiles. For males, there were ten cytokines (IL-1b, MIP-1a, G-CSF, IL-6, IL-1ra, VEGF, IL-10, Eotaxin, MCP1, IFNg) involved in PCA: for females-thirteen (IL-1b, IL-2, IL-13, IL-7, IL-17, Eotaxin, IL-8, IL-10, MIP1a, IFNg, VEGF, IL-1ra, MCP-1).

Machine learning predictions and cytokine ranking: For machine learning analysis, the Orange software package (version 3.26) was utilized. To identify the best algorithms for classifier learning, six different algorithms (Random Forest, Support Vector Machine, Neural Network, Naïve Bayes, Logistic Regression, and Stochastic Gradient Descent) were used. The cytokine profile data were randomly split into the train data set (80%) and the test data set (20%). The training was repeated 20 times. The best algorithms were selected using the area under the curve (AUC) and classification accuracy (CA) parameters. For the sex-based separation of the patient cohort, the best model was identified as Stochastic Gradient Descent, for redox-based stratification, the Support Vector Machine model was selected, and prediction of patient mortality was made using the Naïve Bayes model. The confusion matrix for each algorithm was plotted, and feature importance for each cytokine was calculated as an information gain value.

Statistical analysis: The normality of the data was assessed by Kolmogorov-Smirnov and Shapiro-Wilk tests. Cytokine expression in groups was reported as mean±SEM. Stratified analyses based on cytokine profiles were performed, in which differences in continuous variables were assessed using the Student's t-test for normally distributed data. Correlations were performed utilizing Pearson's or Spearman analyses based on the normality of the data. To visualize high-dimensional data clustering, PCA analysis was carried out by the Orange software package (version 3.26). Kaplan-Meier estimates of patient survival and the hazard ratio for the five-year risk of death were compared between the sexes by a log-rank test. Statistical data analyses were carried out using statistical software, GraphPad Prizm version 8.4. P values<0.05 were considered statistically significant.

### Results

PAH and control cohorts: Table 1 details demographics for both PAH and control cohorts with similar median ages. Both sexes in the PAH cohort showed an equal distribution in functional class, with the most prevalent class III (71% and 68% in males and females, correspondingly). There were no gender differences in six-minute walk distance, brain natriuretic peptide levels, hemodynamic, and cardiac function parameters. Anti-PAH medication profiles were similar in male and female PAH subjects, with approximately 30% treatment-naïve PAH subjects or on PAH mono- and dual therapy (phosphodiesterase inhibitors, endothelin receptor antagonists, or prostanoids). Only ˜10% of PAH subjects were receiving triple therapy. Kaplan-Meier estimates of patient survival showed a lower survival in males, although this difference didn't reach statistical significance (five-year survival rates were 70.1%, CI 79.6-57.6% and 63.3%, CI 77.8-43.6% in female and male patients correspondingly, the hazard ratio (log-rank) was calculated 1.49, CI 0.68-3.31 for females compared with males). In contrast, plasma redox status showed significantly greater oxidative stress in PAH patients of both sexes compared to the sex-matched healthy controls; however, there was no significant difference in the redox profile between the sexes inside the PAH group.

Table 1 shows demographic data and the main clinical parameters of PAH and healthy cohorts. *Healthy controls: Males—n=29, median age 60 yrs (IQR 47-69), median ORP 142 (IQR 123-151); females—n=21, median age 52 yrs (IQR 42-58), median ORP 130 (IQR 126-141). IQR=25-75% interquartile range. #p<0.05 vs. sex-matched healthy subjects.

| | PAH | | | PAH | | | |
|---|---|---|---|---|---|---|---|
| | Males (n = 41) | | N | Females (n = 100) | | N | P value |
| Age*, years, median (25-75% IQR) | 58 | (52-66) | 41 | 61 | (51-70)# | 100 | 0.78 |
| Non-invasive disease metrics | | | | | | | |
| NYHA functional class, n (%) | | | | | | | |
| I | 1 | (2) | | 3 | (3) | | |
| II | 8 | (20) | | 22 | (22) | | |
| III | 29 | (71) | | 68 | (68) | | |
| IV | 3 | (7) | | 7 | (7) | | |
| 6-Minute walk distance (m), median | 364 | (285-414) | 20 | 300 | (206-395) | 68 | 0.1 |
| (IQR) | | | | | | | |
| Brain natriuretic peptide (pg/ml), | 99 | (41-211) | 39 | 117 | (45-298) | 91 | 0.99 |
| median (IQR) | | | | | | | |
| Hemodynamics, median (IQR) | | | | | | | |
| Mean pulmonary arterial pressure | 40 | (32.2-53) | 40 | 40 | (30-49.5) | 96 | 0.56 |
| (mmHg) | | | | | | | |
| Right atrium pressure (mmHg) | 8 | (5-10.5) | 22 | 8 | (4.8-11.3) | 66 | 0.78 |
| Pulmonary vascular resistance (Wood | 5.7 | (2.9) | 40 | 6 | (4) | 93 | |
| units) | | | | | | | |
| Pulmonary artery wedge pressure | 10 | (7-14) | 40 | 10 | (8-14) | 94 | 0.64 |
| (mmHg) | | | | | | | |
| Cardiac output (L/min) | 5.8 | (4.9-6.9) | 40 | 5.7 | (4.5-6.6) | 94 | 0.26 |
| Cardiac index (l/min/m2) | 3 | (2.3-3.4) | 40 | 2.9 | (2.5-3.6) | 94 | 0.64 |
| Cardiac imaging, median (IQR) | | | | | | | |
| Cardiac MRI right ventricle ejection | 32 | (24-40) | 19 | 35 | (25.8-42.7) | 38 | 0.17 |
| fraction (%) | | | | | | | |
| Cardiac MRI right ventricular stroke | 32 | (20.2-40) | 19 | 32 | (26-41) | 39 | 0.34 |
| volume index (mL/m2) | | | | | | | |
| Extent of PAH therapy, n (%) | | | | | | | |
| Treatment naïve | 12 | (29.3) | | 27 | (27) | | |
| Monotherapy | 13 | (31.7) | | 31 | (31) | | |
| Dual therapy | 12 | (29.3) | | 32 | (32) | | |
| Triple therapy | 4 | (9.8) | | 10 | (10) | | |
| Redox status, median (IQR) | | | | | | | |
| Oxidation-reduction potential* | 176.6 | (155-201.8)^{#} | 41 | 179.5 | (145.6-201.4)^{#} | 100 | 0.63 |
| Five-year survival (%), CI | 63.3 | (77.8-43.6) | 30 | 70.1 | (79.6-57.6) | 67 | |
| Hazard ratio (logrank), CI | 0.67 | (0.30-1.48) | 30 | 1.49 | (0.68-3.31) | 67 | 0.30 |

The inflammatory response in PAH: The oxidative-reductive potential (ORP), the primary parameter used to evaluate redox homeostasis, was normally distributed in male and female plasma samples from PAH and healthy subjects. To investigate whether redox status is linked to the inflammatory response, two extreme quartiles were selected, 25% of the most oxidized samples (highest ORP quartile) and 25% of the least oxidized samples (lowest ORP quartile). If both quartiles were combined (plasma redox status is not accounted for), the samples showed a significant increase in cytokines in the PAH cohort. Increases in IL-1b, IL-1ra, IL-2, IL-4, IL-6, IL-7, IL-8, IL-10, IL-12, IL-13, IL-17, G-CSF, IP10, MIP-1a, TNFa, and VEGF were observed in both sexes compared to healthy controls (Table 2). Eotaxin and FGFb were increased in females but were unchanged in males. MIP-1b showed a decrease in males with PAH, but not in females, and RANTES showed a decrease in both sexes. Other cytokines, such as IL-5, IL-9, IL-15, GM-CSF, INFγ, MCP1, and PDGFbb, remained unaltered in each sex compared to healthy subjects.

Table 2 shows cytokine profiles in male and female PAH patients. Multiplex analysis of circulating cytokine panels comprising 27 analytes showed significant upregulation in 18 cytokines and downregulation in 2 cytokines. P values indicate Student t-test analysis of the sex-matched PAH and healthy subjects.

| | Males (PAH vs Control) | | Females (PAH vs Control) | |
|---|---|---|---|---|
| Cytokines | P-value | Changes | P value | Changes |
| IL-1β | p = 0.0002 | ↑ | p = 0.0005 | ↑ |
| IL-1ra | p = 0.0015 | ↑ | p < 0.0001 | ↑ |
| IL-2 | p < 0.0001 | ↑ | p = 0.0036 | ↑ |
| IL-4 | p < 0.0001 | ↑ | p < 0.0001 | ↑ |
| IL-6 | p = 0.0012 | ↑ | p < 0.0001 | ↑ |
| IL-7 | p < 0.0001 | ↑ | p < 0.0001 | ↑ |
| IL-8 | p < 0.0001 | ↑ | p = 0.0002 | ↑ |
| IL-10 | p = 0.0009 | ↑ | p = 0.0004 | ↑ |
| IL-12 (p70) | p = 0.0006 | ↑ | p = 0.0041 | ↑ |
| IL-13 | p < 0.0001 | ↑ | p < 0.0001 | ↑ |
| IL-17 | p < 0.0001 | ↑ | p = 0.0113 | ↑ |
| G-CSF | p < 0.0001 | ↑ | p < 0.0001 | ↑ |
| IP10 | p = 0.0002 | ↑ | p < 0.0001 | ↑ |
| MIP-1α | p < 0.0001 | ↑ | p < 0.0001 | ↑ |
| TNFα | p = 0.001 | ↑ | p = 0.0034 | ↑ |
| VEGF | p = 0.0111 | ↑ | p = 0.0014 | ↑ |
| Eotaxin | p = 0.1533 | | p = 0.0244 | ↑ |
| FGF basic | p = 0.063 | | p = 0.0192 | ↑ |
| MIP-1β | p = 0.0265 | ↓ | p = 0.6608 | |
| RANTES | p < 0.0001 | ↓ | p < 0.0001 | ↓ |
| IL-5 | p = 0.0983 | | p = 0.1219 | |
| IL-9 | p = 0.5715 | | p = 0.3735 | |
| IL-15 | p = 0.775 | | p = 0.2689 | |
| GM-CSF | p = 0.2494 | | p = 0.7414 | |
| IFN γ | p = 0.6449 | | p = 0.084 | |
| MCP1 | p = 0.4719 | | p = 0.845 | |
| PDGF bb | p = 0.446 | | p = 0.2206 | |

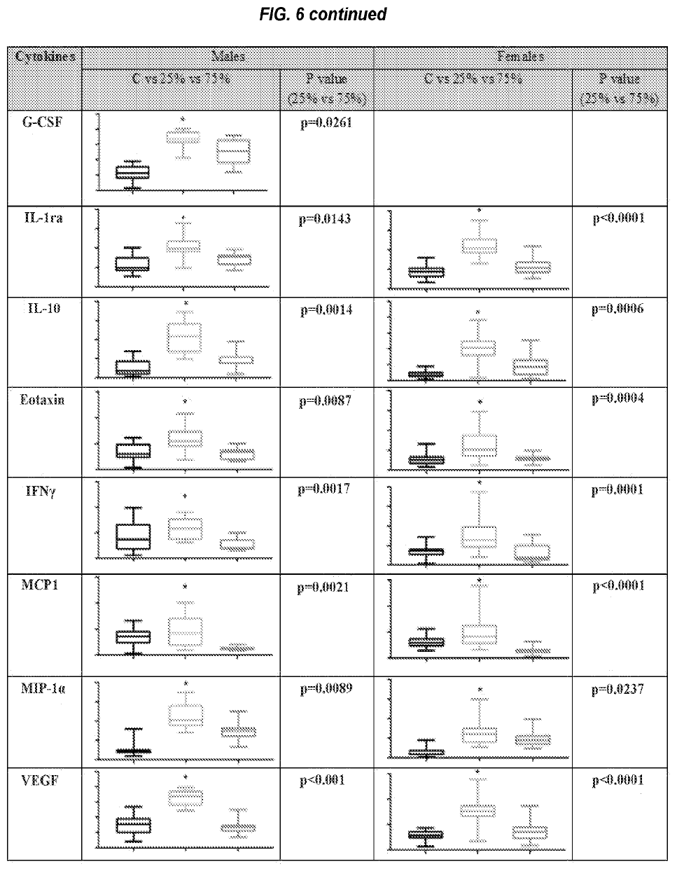

Cytokine profiles with consideration of plasma redox status and patient sex: For consideration of the redox status, the results were compared between the low and high ORP quartiles. FIG. 6 shows a table of cytokines discovered as redox-sensitive since they were found to be significantly altered in one of the extreme redox conditions, either most reduced or most oxidized. Interestingly, some of these redox-sensitive cytokines were not depending on patient sex. Thus, IL-1b was found to be increased only in the most oxidized samples, while IL-1ra, IL-10, Eotaxin, INFy, MCP1, MIP-1a, and VEGF were elevated only in low ORP samples, and these changes were evident in both sexes. In contrast, other cytokines revealed their redox sensitivity only in consideration of sex. Thus, the levels of IL-2, IL-7, IL-13, and IL-17 were increased in the samples with the highest ORP, specifically in women. IL-8 was increased in females' low ORP group, while IL-5, IL-6, IL-15, and G-CSF were also increased in the low ORP group, but only in males. These results suggest that cytokines expression and release may be influenced by the redox state of the microenvironment, although not all cytokines were upregulated by oxidative stress, as commonly expected. Moreover, some cytokines show a possible sex-specific regulation. Thus, female patients have a higher number of cytokines affected by oxidative stress, whereas, in males, all cytokines except IL-1b were upregulated in patients with the least oxidized plasma.

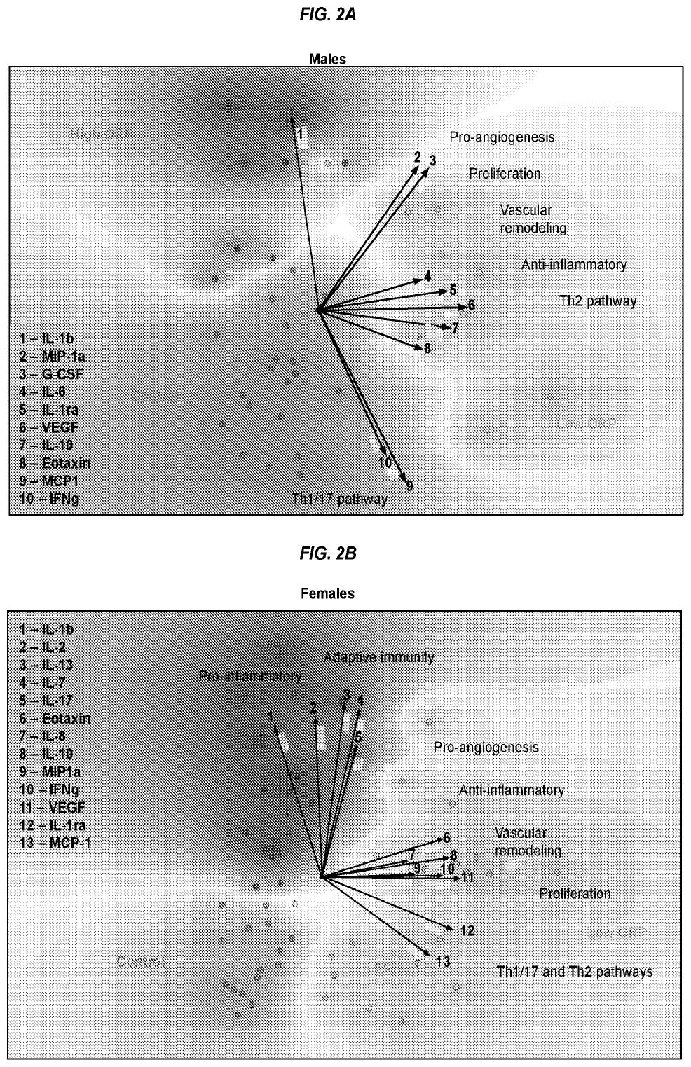

The principal component analysis (PCA) of redox-dependent cytokines showed distinct clustering of control and PAH subjects with low and high ORP status (FIGS. 2A and 2B). Importantly, this separation was achieved only when the data were disaggregated by sex, while unaccounted for sex analysis disrupted the clustering (data not shown). This discovery suggests that the contribution of both factors, sex, and redox status are required to distinguish patients with PAH from healthy controls and could be used for diagnostic purposes. Moreover, the analysis presented in FIGS. 2A and 2B helps to propose particular cytokines as the most influential in the separation of PAH patients from the healthy cohort. In males, IL-1b is the primary determinant of separation of the high-ORP PAH patients from the healthy controls, while MIP-1α, G-CSF, IL-1ra, IL-6, IL-10, VEGF, and Eotaxin all contribute to distinguishing the low-ORP PAH group from controls. In females, cytokines IL-1b, IL-2, IL-7, IL-13, and IL-17 were all involved in the high-ORP group clustering, while Eotaxin, IL-1ra, IL-8, IL-10, VEGF, MIP-1α, IFNγ, and MCP-1 helped to distinguish the low ORP patients.

In both genders, the cytokines profiles were categorized. Pro-inflammatory response mediators were the main factors that defined the patients with a high level of oxidative stress in both sexes. This finding corresponds to the well-established interconnection between oxidative stress and inflammation. However, the mediators of angiogenesis, proliferation, vascular remodeling, and anti-inflammatory pathways were found to contribute to the separation of patients with low ORP (or the less oxidized plasma), suggesting that the low oxidation, or increased level of reduced equivalents, could also be involved in the activation of the pathways associated with PAH initiation and progression.

Correlation between the clinical parameters and the cytokine levels: It was discovered that consideration of sex and/or plasma redox status increases the number of significant correlations. In men, seven cytokines significantly correlated with the changes in the clinical parameters. Except for G-CSF, the elevated cytokine levels corresponded to an increase in the severity of PAH, defined as higher mPAP, PVR, and BNP and lower CO, CI, and 6MWD (Table 3). In women, fourteen cytokines significantly correlated with the severity markers, although only three of them (IL-1b, IL-9, and IP10) positively correlated with the PAH severity. The majority of cytokines, such as IL-2, IL-4, IL-5, IL-7, IL-12, IL-13, IL-15, IL-17, and Eotaxin, correlated with a decrease in PAH severity, suggesting that not an elevated production of these cytokines, but rather their decrease corresponds to more severe disease. It was concluded that in females, cytokines may simultaneously play a role in the PAH progression and the adaptive responses.

Only three out of twenty-one cytokines significantly correlated with the disease parameters in both sexes; two of these, FGFb and INFy, exhibited the opposite effects (a positive correlation with PAH severity in males and a negative correlation in females). Thus, distinct, gender-specific inflammatory profiles differentially contribute to PAH severity.

Table 3 shows a correlation analysis of PAH severity markers and cytokine expression profile. Correlation analysis was done in the PAH cohort disaggregated by sex. A normality test was taken before analysis for each cytokine or clinical parameter. Grey background indicates an increase in PAH severity (defined as higher mPAP, PVR, and BNP; and lower CO, CI, and 6MWD). White background indicates a decrease in PAH severity. Bold p-values indicate significant changes.

| | Males | | | Females | | | Interactions |
|---|---|---|---|---|---|---|---|
| Cytokines | Clin. param. | R | P-value | Clin. para. | R | P value | P-value |
| IL-1β | CI | 0.24 | p = 0.32 | CI | −0.36 | p = 0.017 | p = 0.045 |
| IL-2 | mPAP | 0.30 | p = 0.19 | mPAP | −0.31 | p = 0.0497 | p = 0.04 |
| IL-4 | BNP | 0.05 | p = 0.84 | BNP | −0.396 | p = 0.039 | p = 0.08 |
| IL-5 | 6MWD | −0.16 | p = 0.68 | 6MWD | 0.75 | p < 0.0001 | p = 0.02 |
| | BNP | 0.11 | p = 0.67 | BNP | −0.40 | p = 0.049 | p = 0.16 |
| IL-7 | 6MWD | −0.005 | p = 0.99 | 6MWD | 0.42 | P = 0.02 | p = 0.4 |
| IL-12p70 | mPAP | 0.20 | p = 0.4 | mPAP | −0.36 | p = 0.03 | p = 0.04 |
| | 6MWD | −0.17 | p = 0.63 | 6MWD | 0.44 | p = 0.014 | p = 0.01 |
| IL-13 | mPAP | 0.22 | p = 0.36 | mPAP | −0.40 | p = 0.01 | p = 0.048 |
| IL-17 | RVEF | −0.2 | p = 0.52 | RVEF | 0.53 | p = 0.017 | p = 0.031 |
| Eotaxin | CI | −0.02 | p = 0.93 | CI | 0.31 | p = 0.043 | p = 0.65 |
| | BNP | 0.12 | p = 0.62 | BNP | −0.33 | P = 0.036 | p = 0.13 |
| | 6MWD | −0.15 | p = 0.68 | 6MWD | 0.37 | p = 0.035 | p = 0.71 |
| IL-8 | 6MWD | −0.64 | p = 0.046 | 6MWD | 0.1 | p = 0.6 | p = 0.22 |
| | BNP | 0.64 | p = 0.002 | BNP | 0.1 | p = 0.52 | p = 0.79 |
| MIP-1α | mPAP | 0.45 | p = 0.045 | mPAP | −0.07 | p = 0.66 | p = 0.17 |
| FGF basic | 6MWD | −0.25 | p = 0.48 | 6MWD | 0.39 | p = 0.021 | p = 0.047 |
| | PVR | 0.51 | p = 0.023 | PVR | −0.02 | p = 0.92 | p = 0.046 |
| | mPAP | 0.52 | p = 0.019 | mPAP | −0.19 | p = 0.23 | p = 0.01 |
| IFN γ | PVR | 0.33 | p = 0.19 | PVR | −0.38 | p = 0.013 | p = 0.23 |
| | mPAP | −0.25 | p = 0.31 | mPAP | −0.33 | P = 0.033 | p = 0.49 |
| | BNP | −0.23 | p = 0.36 | BNP | −0.36 | P = 0.024 | p = 0.21 |
| | CO | −0.49 | p = 0.038 | CO | 0.26 | p = 0.1 | p = 0.07 |
| IP10 | 6MWD | 0.75 | p = 0.017 | 6MWD | −0.38 | p = 0.027 | p = 0.001 |
| | CO | 0.09 | p = 0.71 | CO | −0.45 | p = 0.0027 | p = 0.05 |
| | BNP | 0.49 | p = 0.029 | BNP | 0.42 | p = 0.0064 | p = 0.24 |
| | CI | −0.1 | p = 0.69 | CI | −0.35 | p = 0.021 | p = 0.6 |
| | mPAP | 0.56 | p = 0.01 | mPAP | 0.23 | p = 0.13 | p = 0.53 |
| | PVR | 0.42 | p = 0.069 | PVR | 0.37 | p = 0.014 | p = 0.79 |
| IL-9 | CO | −0.15 | p = 0.53 | CO | −0.41 | p = 0.008 | p = 0.49 |
| MIP-1β | CO | −0.46 | p = 0.042 | CO | −0.23 | p = 0.13 | p = 0.95 |
| IL-15 | 6MWD | 0.37 | p = 0.5 | 6MWD | 0.75 | p = 0.006 | p = 0.47 |
| G-CSF | CI | 0.47 | p = 0.044 | CI | 0.25 | p = 0.12 | p = 0.34 |

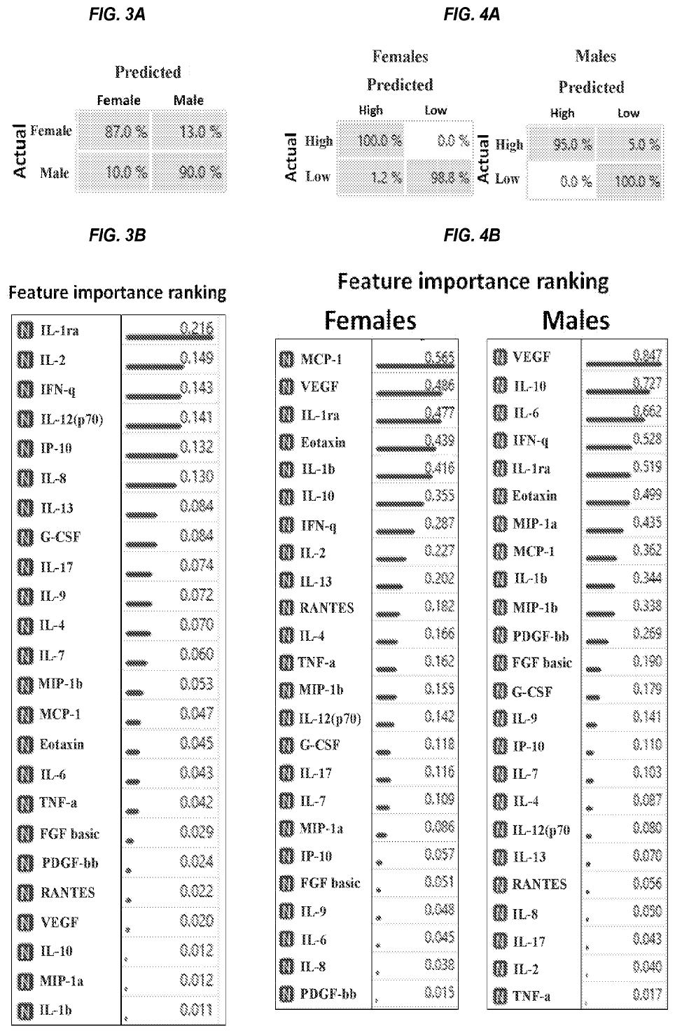

Cytokine profiling-based predictions: To additionally evaluate the potential contribution of sex in the profile of circulating cytokines, the Machine Learning/Deep learning (ML/DL) algorithms were applied. Machine learning models trained to recognize the specific patterns are useful tools to make unbiased predictions of classifications. The confusion matrix shown in FIG. 3A indicates the results of ML predictions of patient sex based on the cytokine profiles. It was found that ML/DL approach can predict the patient's sex with ˜90% accuracy based on the PAH cytokine profile. Although the is no practical use in predicting the sex of the patient, this outcome highlights that the sex-specific profiles of circulating cytokines could be easily identified and separated using ML/DL approach. The ranking of the cytokines shown in FIG. 3B represents the contribution of each cytokine in the sex-specific separation of the overall profile. These results suggest that IL-1ra, IL-2, INFγ, IL-12 (p70), IP10, and IL-8 are the primary influencers that outline the sex difference in the circulating cytokines in PAH.

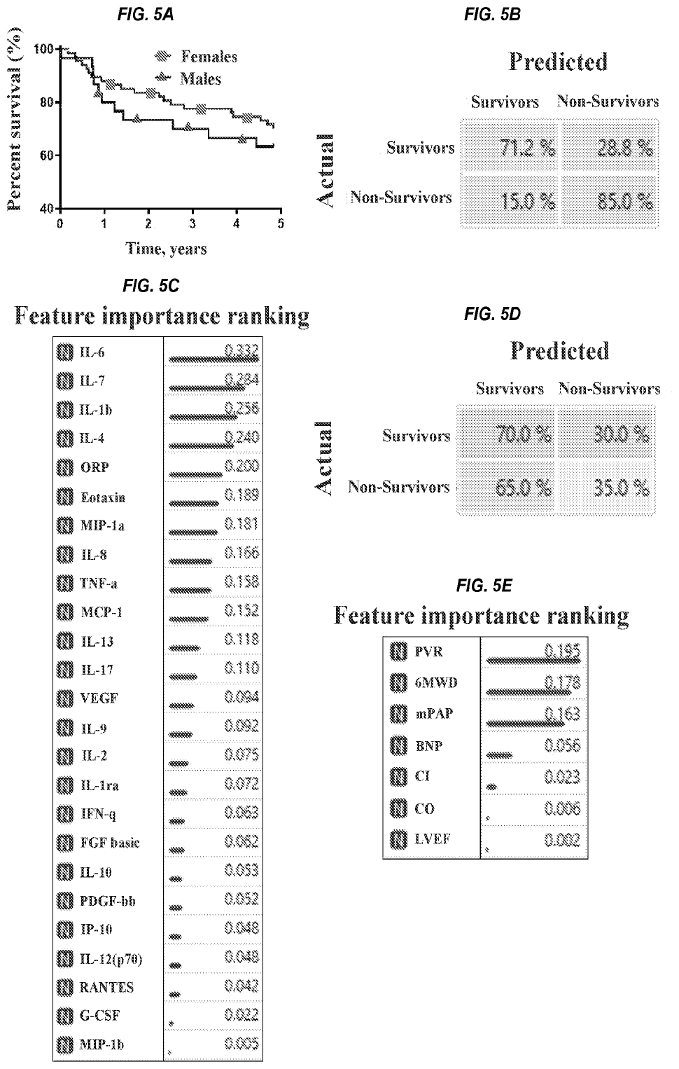

The same ML/DL algorithms were applied to identify the contribution of redox status to the cytokine profile. While no prediction was possible when the analysis was performed in the patients of both sexes (data not shown), the sex-specific approach allowed an accurate (95-100%) prediction of samples with a high or low ORP (FIG. 4A). Again, it was concluded that redox homeostasis significantly contributes to cytokine expression and/or release, although this contribution is sex-specific. Among the cytokines that determine the redox-specific disaggregation of cytokine profile in females are MCP1, VEGF, IL-1ra, Eotaxin, IL-1B, and IL-10, whereas in males—VEGF, IL-10, IL-6, INFγ, IL-1ra, and Eotaxin (FIG. 4B); these are all redox-sensitive cytokines (FIG. 2A-2B), which explicitly increased in the low-ORP samples, except for IL-1β (FIG. 6).

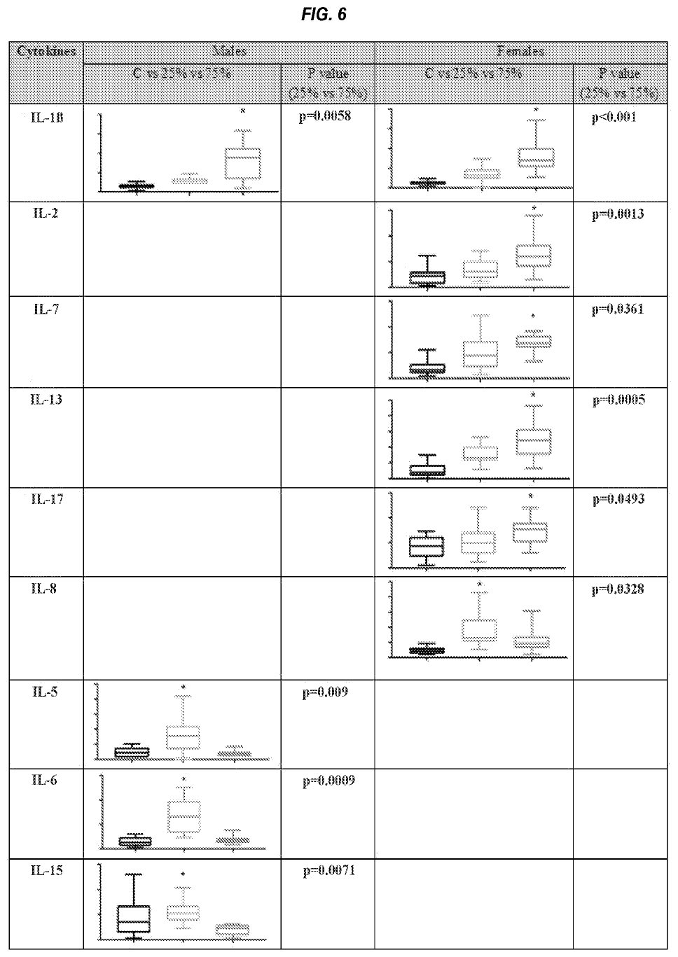

Finally, the ML/DL approach was applied to predict patient survival. Compared to the previous analysis done to validate the contribution of sex and redox status in cytokine profiling, this type of prediction is of high importance, as there is a demanding need to identify the patients at a high risk of mortality. The five-year survival in the PAH cohort was 70.1% (CI 79.6-57.6%) in females and 63.3% (CI 77.8-43.6%) in male patients (FIG. 5A). The combined cytokine and ORP profiles allowed an accurate statistical classification of survivors vs. non-survivors. As shown in the confusion matrix (FIG. 5B), the episodes of mortality were predicted with 85% accuracy. However, the same predictive analysis applied for the primary clinical parameters showed a much higher confusion of the model with accuracy in predicting patient mortality only 35% (FIG. 5D). Although cytokine and clinical markers profiles showed a comparable accuracy for predicting patient survival, the profiling of circulating cytokines could become a useful tool specifically for predicting the episodes of patient mortality. The cytokines that showed the highest rank in predicting the outcome were IL-6, IL-7, IL-1β, IL-4, Eotaxin, and MIP1β (FIG. 5B). Notably, the ORP was found among the highest rank factors, suggesting the critical importance of the plasma redox status in patient survival. Among the most efficient in separating survivors vs. non-survivors clinical markers were PVR, 6MWD, and mPAP.

In the present study, two criteria were applied to stratify the initial PAH cohort. First, male and female samples were discretely analyzed and then patients were further divided based on the redox status of plasma. Furthermore, the separation of patients by plasma redox status allows comparing the contribution of necrotic cell death, which shifts plasma toward less oxidized (low-OPR), or the oxidative stress, which increases oxidation of plasma (high ORP), to the severity of PAH. Indeed, some cytokines known to be produced in response to necrosis but not apoptosis were increased only in males and only in low-OPR samples. Moreover, in each sex, the samples with high and low ORP were clustered differently, although both exhibited a strong separation from the healthy cohort. Based on these results, it was proposed that plasma redox homeostasis may represent an important contributor to sub-phenotyping of PAH patients and be implemented into underlying pathology. Moreover, this study outlines the cytokines that displayed redox-sensitivity, as they were found to be significantly elevated in one of the extreme redox conditions—in plasma with the highest or lowest level of oxidation. Although the large body of published literature confirms the increased oxidative stress in the area of inflammation, the particular cytokines which expression depends on the severity of oxidative stress were never identified.

While oxidative stress stimulates cytokine production, it is also involved in the “sterilization” of the intracellular content in apoptotic cells, making this type of death immune-silent. Conversely, necrotic cell death induces a significant inflammatory response mediated by damage-associated molecular patterns (DAMPs) spilled out of necrotic cells. This inflammatory reaction could occur together with the redox shift toward less oxidized due to the release of reducing equivalents from damaged cells. Therefore, the production of some cytokines may correspond to the less oxidized conditions. Our data indicate that IL-1b is a markedly oxidative stress-driven cytokine that achieves the highest expression in an oxidative environment in both male and female patients. Other cytokines that showed increased expression in a highly oxidative milieu are IL-2, IL-7, IL-13, and IL-17, all showing strong proinflammatory characteristics. The remaining cytokines are increased in the less oxidized milieu, suggesting that the less oxidized environment is more favorable for cytokine production in PAH.

The difference in the redox homeostasis for each sex and the sex-specific correlations between the clinical parameters and circulating cytokines also highlight the importance of sex as a factor separating the PAH cohort on sub-groups. In males, most cytokines positively correlated with the PAH severity, as it was defined earlier (higher mPAP, PVR, and BNP, and lower CO, CI, and 6MWD). The pro-inflammatory properties of cytokines promoting PAH in males suggest the importance of an inflammatory component for this sex in PAH severity. For example, IL-8, which significantly correlates with a decrease in 6MWD and increase in BNP, is a major neutrophil chemoattractant released by pulmonary vascular cells, lung epithelium, and macrophages (31). Attracted to the lungs, neutrophils can perpetuate the inflammatory response by releasing cytokines, proteases, ROS and producing secondary damage to the surrounding tissue.

As used herein, the term “about” refers to plus or minus 10% of the referenced number. Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A computer-implemented method for diagnosing and prognosing a subject with a disease, medical screening, and monitoring therapy efficacy, the method comprising:
a) inputting into a computer system quantitative data of a panel of biomarkers in a biological sample obtained from the subject;
b) analyzing the quantitative data with machine learning or deep learning models or their ensembles;
c) using a first-tier biomarker multi-panel to distinguish healthy subjects from subjects with one or more diseases that affect different organs or cell types, said biomarker multi-panel previously determined by using a selection of biomarkers executed on a plurality of clinical parameters;
d) determining and using a second-tier biomarkers panel that can implement machine learning, deep learning algorithms, or a combination thereof to sub-phenotype the one or more diseases of the organ or the cell type affected identified in step c; and
e) diagnosing or prognosing the subject if the quantitative data of the panel of biomarkers in the biological sample obtained from the subject is correlated by the computer system using tiered panels and machine learning, deep learning algorithms, or a combination thereof to produce risk scores or other values that are indicative of the one or more diseases.

2. The method of claim 1, wherein internal or external standards are used in the acquisition of the quantitative data.

3. The method of claim 1, wherein the method additionally comprises determining and using a third-tier biomarkers panel that can implement machine learning, deep learning algorithms, or a combination thereof to identify specific etiology or comorbidities of the one or more diseases of the organ or the cell type affected identified in step c.

4. The method of claim 1, wherein the biomarker selection is based on statistical significance, pathology of disease by an expert-in-the-loop, feature selection optimization, or a combination thereof, and wherein feature selection optimization uses machine learning, deep learning algorithms, or a combination thereof.

5. The method of claim 4, wherein the feature selection optimization has been trained using a quantity of a panel of biomarkers from subjects having the disease and from control subjects that do not have disease.

6. The method of claim 1, wherein the quantity of the panel of biomarkers is determined using standard clinical chemistry techniques, protein analytic techniques, nucleic acid techniques, and/or analytical techniques suitable for metabolite analysis.

7. The method of claim 6, wherein the techniques comprise gas chromatography (GC) coupled to mass spectrometry (GC-MS), liquid chromatography-mass spectrometry (LC-MS), other mass spectrometry methods, or nuclear magnetic resonance (NMR).

8. The method of claim 7, wherein the clinical parameters comprise sex, plasma redox status, and cytokine levels.

9. The method of claim 1, wherein the trained machine learning and deep learning algorithms comprise linear regression, logistic regression, decision tree, support vector machine, Naive Bayes, K nearest neighbors, K-Means, random forest, artificial neural networks, or a combination thereof.

10. The method of claim 1, wherein the metabolites comprise carbohydrates, amino acids, fatty acids, and/or nucleotides and their intermediates or derivatives.

11. The method of claim 1, further comprising steps for preparing the quantitative data of the panel of metabolic biomarkers for inputting into the computer system, the steps comprising:
a) labeling the quantitative data with one or more confirmed diagnoses of a pathological condition;
b) applying a plurality of characteristics of the patient to the quantitative data;
c) balancing the dataset through exclusion of data that does not correspond to a disease biomarker, addition of multiple-use data points, or a combination thereof; and
d) scaling the dataset to a fixed range.

12. The method of claim 11, wherein the plurality of characteristics comprises gender, age, race, ethnicity, time and date of sample collection, and patient condition at the time and date of sample collection.

13. The method of claim 11, wherein the excluded data comprises metabolites associated with consumption of certain food or drugs, redundant metabolites, and metabolites that contribute to noise.

14. The method of claim 11, wherein the multiple-use data points comprise randomly picked data points with an underrepresented label.

15. The method of claim 11, wherein the dataset is scaled to a range of [0, 1].

16. A non-transitory, computer-readable medium having computer-executable instructions for causing a processor to execute a method for diagnosing a subject with a disease, the method comprising:
a) determining whether quantitative data of a panel of metabolic biomarkers in a biological sample obtained from the subject is indicative of the disease using a trained machine deep learning classifier for distinguishing subjects with different diseases and without disease;
wherein the machine deep learning classifier has been trained using quantitative data of a panel of metabolic biomarkers from subjects having the disease and from control subjects that do not have disease; and
b) diagnosing the subject if the quantitative data is determined by the machine deep learning classifier to be indicative of the disease.

17-19. (canceled)

20. A non-transitory, computer-readable medium having computer-executable instructions for training a multi-label machine learning model to identify disease biomarkers in a patient, the computer-executable instructions comprising:
a) computationally selecting one or more profiles, wherein each profile is selected from a group comprising metabolomic profiles, proteomic profiles, or a combination thereof;
b) computationally selecting, for each profile of the one or more profiles, one or more change-disease relationships between a change to the profile and one or more disease biomarkers that induce the change;
c) providing a structural model for each change-disease; and
d) processing, by at least a first tier of the machine learning model, each structural model such that the machine learning model is trained to identify, based on a change to a profile of the patient, the one or more disease biomarkers that induced the change.

21. The non-transitory, computer-readable medium of claim 20 further comprising computer-executable instructions comprising:
a) computationally selecting, for each disease biomarker selected in step b of claim 20, one or more disease-etiology relationships between the disease biomarker and one or more etiologies of the disease biomarker;
b) providing a structural model for each disease-etiology relationship; and
c) processing, by at least a second tier of the machine learning model, each structural model such that the machine learning model is trained to identify, based on the one or more changes to the profile of the patient and the one or more disease biomarkers identified in the patient, the one or more etiologies of the one or more disease biomarkers.

22. The non-transitory, computer-readable medium of claim 20 further comprising computer-executable instructions comprising:
a) computationally selecting, for each disease biomarker selected in step b of claim 20, one or more disease-comorbidity relationships between the disease biomarker and one or more comorbidities associated with the disease biomarker;
b) providing a structural model for each disease-comorbidity relationship; and
c) processing, by at least a second tier of the machine learning model, each structural model such that the machine learning model is trained to identify, based on the one or more changes to the profile of the patient and the one or more disease biomarkers identified in the patient, the one or more comorbidities of associated with the one or more disease biomarkers.

23-35. (canceled)

36. The method of claim 3, wherein the method further comprises tracking, by the third-tier biomarkers panel, a progression of the one or more diseases over time to determine the efficacy of the treatment.
